(19) United States
(12) Patent Application Publication
(10) Pub. No.: US 2011/0270034 A1
(43) Pub. Date: Nov. 3, 2011

(54) ENDOTRACHEAL TUBE WITH SIDE MOUNTED CAMERA AND ILLUMINATOR

(76) Inventor: Robert A. Mackin, Flagstaff, AZ (US)

(21) Appl. No.: 13/107,161

(22) Filed: May 13, 2011

Related U.S. Application Data
(63) Continuation-in-part of application No. 11/865,256, filed on Oct. 1, 2007, now Pat. No. 7,942,813.
Application No. 11/865,256 is a division of application No. 10/775,904, filed on Feb. 10, 2004, now Pat. No. 7,297,105.

(51) Int. Cl. A61B 1/05

(52) U.S. Cl. 600/112

(57) ABSTRACT

An endotracheal tube includes an inflatable balloon close to the distal end. A camera is mounted on the wall of the endotracheal tube proximate and upstream from the balloon to provide a real time image of any accumulation of secretions in the area. A light emitting diode is also mounted in the wall of the endotracheal tube to provide illumination for the camera. Sets of electrical conductors may be embedded in the wall of the endotracheal tube to provide electrical power to the camera and to the light emitting diode and to convey a signal from the camera representative of the image captured by the camera. Connectors attached to the electrical conductors are mated with a transmitter module housing electrical power (such as batteries) for providing electrical power to the light emitting diode and to the camera and electrical circuitry for transmitting the signal from the camera with a radio frequency transmitter. A remotely located radio frequency receiver receives the transmitted signal and is coupled with a video monitor to display the real time image captured by the camera.

### CROSS-REFERENCE TO RELATED APPLICATIONS

This application is a continuation-in-part of a patent application entitled “Detachable Endotracheal Camera” filed Oct. 1, 2007 and assigned Ser. No. 11/865,256, now U.S. Pat. No. 7,942,813, which is a divisional of a patent application entitled “Endotracheal Camera”, filed Feb. 10, 2004 and assigned Ser. No. 10/775,904, now U.S. Pat. No. 7,297,105, both of which describe inventions by the present inventor.

### BACKGROUND OF THE INVENTION

1. Field of the Invention

The present invention relates to endotracheal tubes and, more particularly, to an endotracheal tube having an inflatable balloon with a light emitting diode and a camera attached to the wall of the endotracheal tube adjacent and upstream of the balloon for display of any secretions present on a video monitor.

2. Description of the Prior Art

The basic tenets attendant endotracheal tubes having an illuminator at the distal end are illustrated and described in U.S. Pat. No. 5,285,778 and relating to an invention by the present inventor; which patent is incorporated herein by reference. The endotracheal tube described therein includes an optical fiber extending through the endotracheal tube to a viewing lens at the distal end of the tube. An eye piece is attached to the proximal end of the optical fiber to permit viewing through the lens. Illumination of the area under inspection is provided by a high intensity light source extending via the endotracheal tube to an illumination port at the distal end.

### SUMMARY OF THE INVENTION

An endotracheal tube includes an inflatable balloon disposed proximal the distal end of the endotracheal tube. A light emitting diode is mounted on the wall of the endotracheal tube upstream of the balloon to provide illumination of the adjacent tissue and any secretions present. A camera also mounted on the wall of the endotracheal tube proximate the light emitting diode provides an image of the illuminated area. Electrical conductors, which may be embedded in the wall of the endotracheal tube, extend from the light emitting diode and the camera to provide electrical power and to convey a signal from the camera reflective of the captured image. A plug with one or more sets of prongs is at the terminal end of the electrical conductors for engagement with electrical components in a transmitter module to provide electrical power to the light emitting diode and to the camera and to transmit the signal from the camera with a low power transmitter. A receiver receives the transmitted signal and conveys it to a video monitor for display of the image captured by the camera.

It is therefore a primary object of the present invention to provide a method for real time viewing of secretions or the conditions of the trachea that may be present.

Another object of the present invention is to provide an endotracheal tube mounted camera and light emitting diode for recording an image of secretions or conditions that may be present in the trachea upstream of the balloon forming a part of the endotracheal tube.

A yet further object of the present invention is to provide a camera and light emitting diode embedded in the wall of an endotracheal tube and electrical conductors extending therefrom along the endotracheal tube to be coupled with a transmitter for transmitting an image captured by the camera for display on a remote video monitor.

Still another object of the present invention is to provide electrical conductors supported by an endotracheal tube for coupling a light emitting diode and a camera embedded in the wall of an endotracheal tube with a transmitter for transmitting the image captured by the camera to a receiver for display real time on a video monitor.

A further object of the present invention is to provide a light emitting diode and a camera in the wall of an endotracheal tube and electrical conductors extending therefrom to a remote transmitter for transmitting an image captured by the camera to a video monitor for display.

A yet further object of the present invention is to provide a light emitting diode and a camera mounted upstream of the balloon of an endotracheal tube for conveying an image through electrical conductors selectively coupled to apparatus for displaying a captured image.

A still further object of the present invention is to provide an apparatus for displaying an image real time on a video monitor by capturing the image to be displayed with a camera and light emitting diode mounted on the wall of an endotracheal tube upstream of the balloon and transmitting the image with a radio frequency transmitter to a corresponding receiver to produce a signal reflective of the captured image for the video monitor.

A still further object of the present invention is to provide an apparatus for viewing on a video monitor in real time an image of any secretions proximate the balloon of an endotracheal tube using essentially an off the shelf low cost camera, light emitting diode and a wireless transmitter and receiver.

These and other objects of the present invention will become apparent to those skilled in the art as the description thereof proceeds.

### BRIEF DESCRIPTION OF THE DRAWINGS

The present invention will be described with greater specificity and clarity with reference to the following drawings, in which:

FIG. 1 is a partial cross-sectional view of an endotracheal tube embodying fiber optics for transmitting a signal;

FIG. 2 is a partial cross-section of the endotracheal tube;

FIG. 3 is a partial cross-sectional view illustrating placement within a patient of an endotracheal tube;

FIG. 4 illustrates a camera and a transmitter for attachment with a connector of an endotracheal tube;

FIG. 5 illustrates a receiver and an attached video monitor;

FIG. 6 is a partial cross-sectional view of an endotracheal tube embodying the present invention;

FIG. 7 is a representative cross-sectional view of the endotracheal tube; and

FIG. 8 illustrates a transmitter, receiver and a video monitor.

### DESCRIPTION OF THE PREFERRED EMBODIMENT

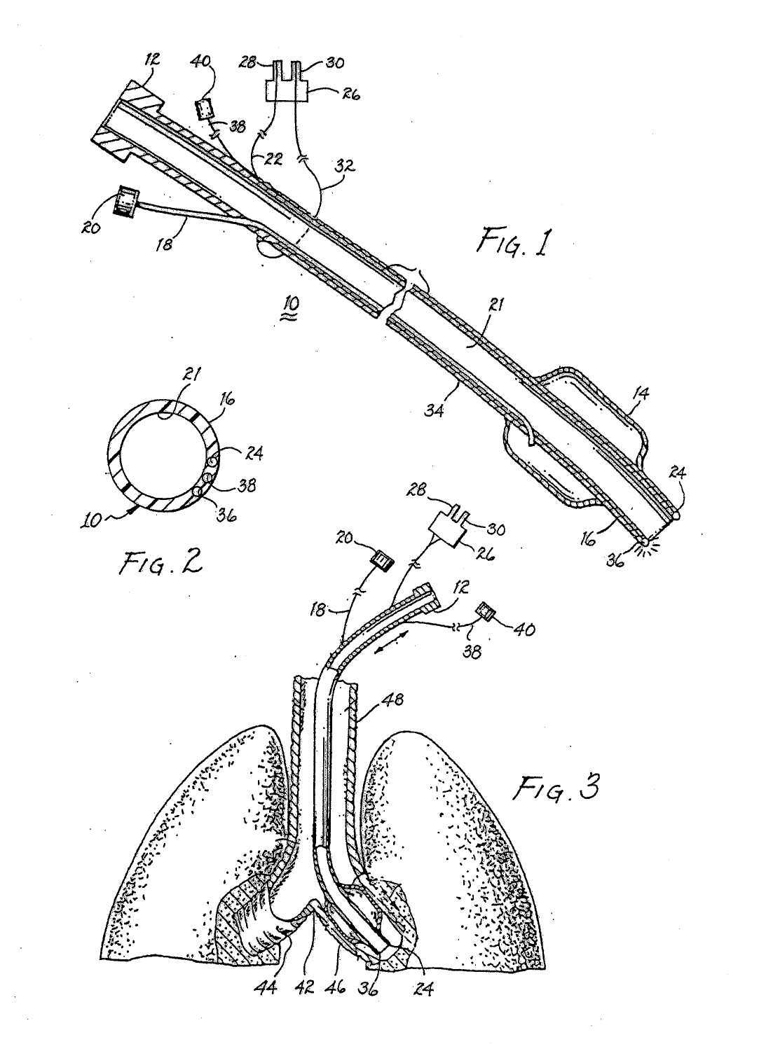

Referring to FIG. 1, there is shown an endotracheal tube 10 having a connector 12 for connection to a conventional ventilator to assist a patient's breathing function. The endotracheal tube includes an inflatable balloon 14 in proximity to its distal end 16. The inflatable balloon is inflated by a tube 18 connected through a connector 20 to a small syringe-like air pump after the endotracheal tube has been inserted into a patient's trachea.

Prior endotracheal tubes do not permit any visualization of a patient's tracheal and bronchial passages. If such visualization is needed, connector 12 is disconnected from the ventilator and a conventional bronchoscope is inserted down through hollow passage 21 of the endotracheal tube to allow a physician to determine if a lot of mucus is present in either lung or in either of the left or right stem main bronchi. If it is necessary to suction mucus out of either of the patient's lungs, a suctioning tube is inserted through hollow passage 21. The endotracheal tube may have to be disconnected from the ventilator to allow visualization in the trachea of the lungs or to allow suctioning of the mucus, blood, etc. if the endotracheal tube does not have a sealable side port through which the suctioning tube can be inserted.

When a skilled physician, often a pulmonologist, inserts an endotracheal tube into a patient, it would be desirable for a nurse to be able to easily monitor the position of the endotracheal tube in a patient's trachea to determine if its location has been shifted. If so, the nurse would know whether to call a physician to reposition the endotracheal tube. It would also be desirable to determine accurately the position of the endotracheal tube without requiring an x-ray of the patient.

Still referring to FIG. 1, endotracheal tube 10 includes an optical fiber, hereinafter referred to as fiber optic bundle 22, that extends through the endotracheal tube to a viewing lens 24 at distal end 16. The fiber optic bundle can be an inexpensive plastic optical fiber costing only a few dollars and embedded in the wall of the endotracheal tube. The fiber optic bundle is operatively connected to a connector 26 which includes two prongs 28, 30 of which prong 28 carries the fiber optic bundle. A second plastic optical fiber, hereinafter referred to as fiber optic bundle 32, extends through wall 34 of endotracheal tube 10 to an illumination port 36 at distal end 16.

FIG. 2 is a view of the distal end of endotracheal tube 10. A hollow tube 38 extends from a flushing inlet port connector 40 (see FIG. 1) and extends through the endotracheal tube so that a transparent saline flushing liquid can be forced through the tube to wash mucus away from viewing lens 24 and illumination port 36. Such mucus may collect thereon during insertion of the endotracheal tube into the patient's trachea or afterward.

One major advantage of endotracheal tube 10 is that the carina (a cartilaginous structure) 42 (see FIG. 3) can be easily viewed during insertion of the endotracheal tube so that a nurse or a physician can readily determine how far into the patient's trachea to properly insert the endotracheal tube. This avoids the need for an x-ray process to determine if the endotracheal tube is properly inserted. As the endotracheal tube can become malpositioned in the patient and which would normally require a later x-ray to check for proper placement, direct visualization can avoid the need for such a repeat x-ray. Another advantage is that the nurse or physician can easily view the conditions in branches 44, 46 of trachea 48 to determine the presence of mucus or other condition and to determine whether there is a need for immediate suctioning of mucus, blood, etc., from either lung or the passages thereto.

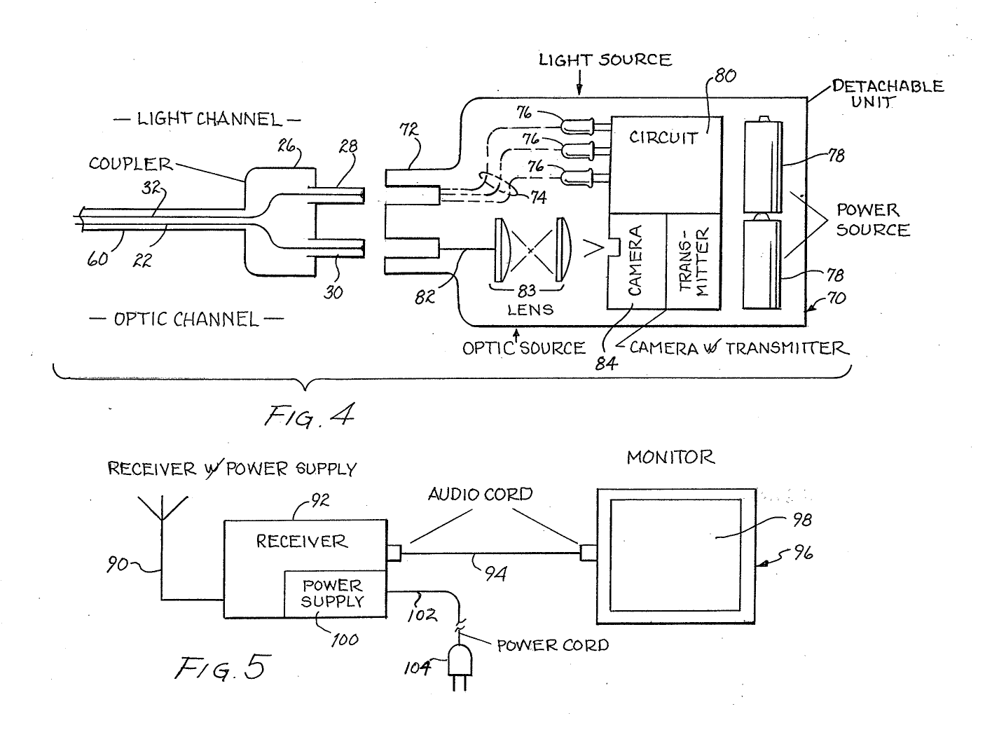

Referring to FIG. 4, there is shown a male connector 26 having prongs 28, 30 extending therefrom. Fiber optic bundle 32 is in functional and operative engagement with prong 28 to transmit light from the end of the prong to illumination port 36 at distal end 16 of the endotracheal tube. Fiber optic bundle 22 is coupled with lens 24 at the distal end of the endotracheal tube to transmit light, that is an image, to the end of prong 30. As illustrated, fiber optic bundles 22 and 32 may be incased within a sheath 60.

A removable module 70 includes a female connector 72 for receiving prongs 28, 30 of connector 26. Upon mating of connectors 26, 72, fiber optic bundle 32 within prong 28 is placed in communication with fiber optic bundle 74, the latter being in communication with and receiving light from light emitting diodes 76. Electrical power for the light emitting diodes is provided by circuit 80 connected to batteries 78. Prong 30 of male connector 26 mates with female connector 72 to transmit light, that is, the image visible through lens 24 (see endotracheal tube 10) to convey the received light through a further fiber optic bundle 82 to a lens system 83. The lens system is interconnected with a small sized and relatively inexpensive electronic camera 84. Cameras suitable for this purpose cost less than $100.00 and can be found for less than $50.00 from commercial outlets. The camera is interconnected with a low power radio frequency transmitter 86 to transmit the images recorded by the camera. Transmitters of this type are readily available for less than $100.00 and may be found for less than $50.00 from commercial outlets.

As shown in FIG. 5, an antenna 90 is connected to a radio frequency receiver 92 and receives the images detected by camera 84 and transmitted by transmitter 86. The received image is conveyed via an electrical conductor or cord 94 to a video monitor 96. The video monitor includes a screen 98 for displaying the image recorded by camera 84. As illustrated, a power supply 100 provides power to receiver 92 and to video monitor 96 through an electrical conductor. Power to the power supply may be provided by an electrical conductor connected to a conventional plug 104 for engagement with a conventional wall socket.

In summary, the image conveyed from the lens at the distal end of the endotracheal tube is digitized and recorded by a camera. The image recorded by the camera is displayed real time on a video monitor through a wireless interconnection. The ease of a wireless transmission system in the confines of an operatory avoids the likelihood of a patient and attending health care providers from becoming entangled with cords and wires.

Moreover, presently used wires and cables extending to a video monitor creates a hazard of an attending health care provider inadvertently interfering with such wires and/or cables and causing repositioning or pulling out of the endotracheal tube. This hazard is completely avoided by the present invention due to the absence of such wires and/or cables.

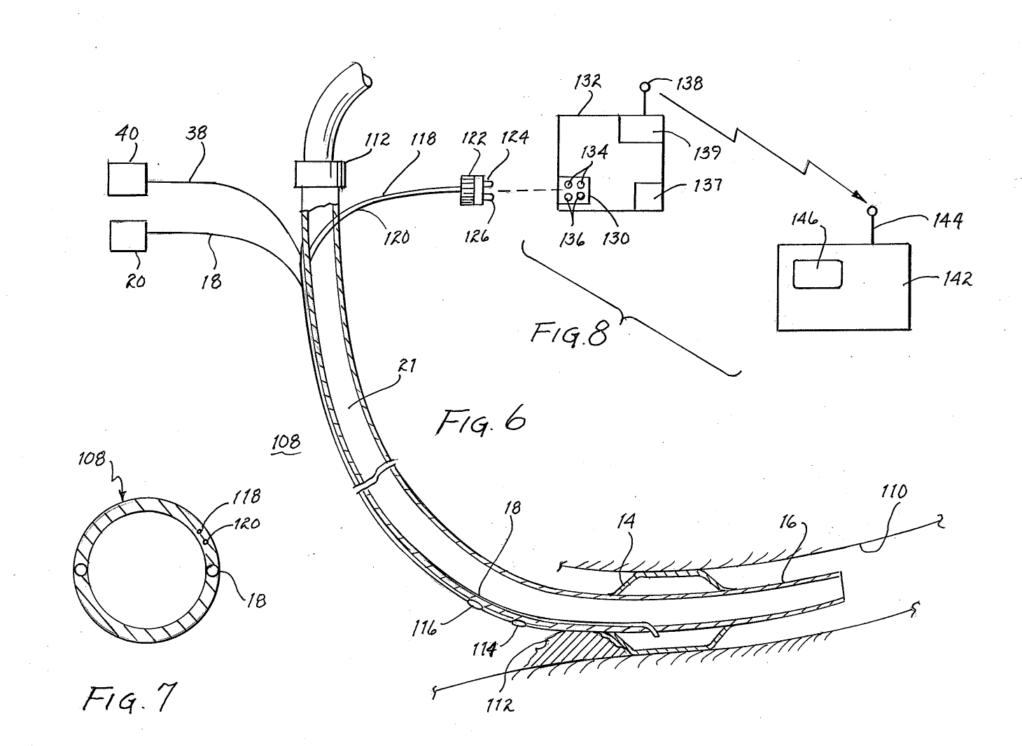

Referring to FIG. 6, there is illustrated an endotracheal tube 108 for insertion such as within trachea 110 of a patient. The endotracheal tube may be flexible or rigid, depending upon the procedure to be performed. Upstream of distal end 16 of the endotracheal tube is an inflatable balloon 14. A connector 20 is connectable to a source of air/gas under pressure for the purpose of inflating balloon 14 through tube 18, which tube extends into the balloon, as illustrated. After inflation of the balloon for purposes of ventilating, secretions may collect between the wall of trachea 110 and the endotracheal tube upstream of balloon 14. Previously, it was impossible to determine the presence of and removal of such secretions by any simple procedure. Accordingly, a suction tube was sometimes used to withdraw any secretions that may have accumulated whether or not there actually had been an accumulation of secretions. Necessarily, this procedure may be carried out by happenstance and is disruptive of the actual medical procedure being undertaken. By attaching a camera 114 to the wall of endotracheal tube 108 upstream from balloon 14, it is possible to obtain a real time image depicting the presence or absence of accumulation of secretions 112 or other conditions in the trachea above the balloon. A light emitting diode 116 is also attached to the wall of the endotracheal tube to provide illumination of the secretions, if present, and conditions in the surrounding area so that an image of the area proximate camera 114 can be obtained.

A first set of electrical conductors 118 extends from light emitting diode 116 to convey electrical energy for operation of the light emitting diode. A second set of electrical conductors 120 extends from camera 114 to provide both electrical power for operation of the camera and for conveying a signal reflective of the image captured by the camera. These two sets of electrical conductors may terminate at an electrical connector 122 having a first set of prongs 124 coupled with first set of conductors 118 and a second set of prongs 126 coupled with second set of conductors 120. As illustrated in FIG. 7, first and second sets of conductors may be embedded within the wall of endotracheal tube 108. Alternatively, they may be attached adjacent the interior or exterior wall surface of the endotracheal tube, depending upon manufacturing and cost considerations.

One of the purposes of the present invention is to provide a relatively inexpensive endotracheal tube which has the capability of providing an image of an area of interest with a camera located in proximity to such area. This is presently achievable as cameras sized approximately one millimeter in cross-section and at a cost of less than $100.00 are commercially available. Additionally, light emitting diodes of approximately one millimeter in cross-section which provide sufficient illumination to illuminate an area of interest are commercially available. Their cost is also significantly less than $100.00. By using sets of electrical conductors supported by the endotracheal tube itself, essentially no additional bulk results from such mounting of the sets of electrical conductors.

As shown in FIG. 8, to reduce the presence of cables, wires, etc. proximate the area where the medical procedure is being performed, connector 122 is connectable with a mating connector 130 mounted within a transmitter module 132 of relatively small size. Connector 130 includes, for example, a pair of female receptacles 134 to receive first set of prongs 124 and a further pair of female connectors 136 to receive second set of prongs 126. The function and purpose of transmitter module 132 is that of providing power to the camera and the light emitting diode and to transmit a signal reflective of the image captured by the camera. As the energy consumption of the camera and the light emitting diode are very modest, conventional commercially available batteries 137 of small size and capacity can be housed within transmitter module 132 for this purpose.

Additionally, the housing includes a transmitter 139 of low power and hence low power consumption, for transmitting via antenna 138 a signal representative of the signal generated by camera 114 corresponding with the image captured by the camera. The receiver 142, including an antenna 144 for receiving the signal transmitted by antenna 138, may be located at a location remote from the transmitter module. The receiver includes or is coupled to a video monitor 146 to provide an image corresponding with the image captured by camera 114.

As medical personnel present during a medical procedure involving endotracheal tube 108 are undertaking the medical procedure, secretions 112 may accumulate upstream of balloon 14. Simultaneously, the accumulation or presence of secretions 112 will be continuously displayed on video monitor 146. If present, suction apparatus can be used in trachea 110 to remove such secretions on an on-going basis or upon accumulation of a certain quantity of secretions. It is therefore evident that both the accumulation of secretions as well as the removal of the secretions will be captured by camera 114 and displayed on video monitor 146 to provide the medical personnel with the real time image of the area wherein secretions are expected while the medical procedure is being performed.

Although the use of a light emitting diode to provide illumination may be preferred, lighting the area of interest could also be provided by one or more fiber optic cables, as described above with respect to FIGS. 1, 2 and 4.

I claim:

1. Apparatus for displaying an image of secretions in the trachea proximate the upstream end of a balloon of an endotracheal tube, said apparatus comprising:
(a) a source of light disposed in the wall of said endotracheal tube upstream of said balloon for illuminating the tissue to be imaged, said source of light comprising at least one light emitting diode;
(b) a low cost camera disposed in the wall of said endotracheal tube proximate said source of light for generating an electrical signal representative of an image captured by said camera;
(c) a first set of electrical conductors extending from said source of light to a first electrical connector;
(d) a second set of electrical conductors extending from said camera to a second electrical connector;
(e) a low cost transmitter module adapted for electrical connection with said first and second electrical connectors to provide electrical power to said source of light and to said camera and a low cost radio frequency transmitter for transmitting the signal generated by said camera;
(f) a low cost radio frequency receiver for receiving said signal; and
(g) a video monitor coupled with said receiver for displaying the image represented by the signal received by said receiver.

2. The apparatus as set forth in claim 1 wherein said endotracheal tube is a flexible tube.

3. The apparatus as set forth in claim 1 wherein said endotracheal tube is a rigid tube.

4. The apparatus as set forth in claim 1 wherein said first and second electrical conductors extend along said endotracheal tube.

5. The apparatus as set forth in claim 4 wherein a part of the length of each of said first and second electrical conductors is embedded in the side wall of said endotracheal tube.

6. Apparatus for displaying an image of substance in the trachea upstream of the balloon of an endotracheal tube, said apparatus comprising in combination:
(a) a source of light disposed adjacent the wall of said endotracheal tube for illuminating the substance to be imaged;
(b) a camera disposed adjacent the wall of said endotracheal tube proximate said source of light for recording an image of any substance present;
(c) electrical conductors extending from said source of light along said endotracheal tube;
(d) further electrical conductors extending from said camera along said endotracheal tube;
(e) a transmitter module for providing electrical power to said electrical conductors and said further electrical conductors and for receiving a signal from said camera reflective of an image recorded by said camera and for transmitting the signal;
(f) a receiver for receiving the transmitted signal; and
(g) a display for receiving the transmitted signal from said receiver and for displaying an image represented by the transmitted signal.

7. The apparatus as set forth in claim 6 wherein said endotracheal tube is rigid.

8. The apparatus as set forth in claim 6 wherein said endotracheal tube is flexible.

9. The apparatus as set forth in claim 6 wherein said source of light comprises at least one light emitting diode.

10. The apparatus as set forth in claim 6 wherein a length of each of said electrical conductors and said further electrical conductors is embedded in the wall of said endotracheal tube.

11. The apparatus as set forth in claim 6 including mating electrical connectors for detachably attaching said electrical conductors and said further electrical conductors to said transmitter module.

12. A method for detecting the presence of a foreign substance upstream of the balloon of an endotracheal tube, said method comprising the steps of:
(a) illuminating the tissue and any foreign substance upstream of the balloon with at least one light emitting diode;
(b) capturing an image of the tissue and any foreign substance upstream of the balloon with a camera;
(c) providing electrical power to the at least one light emitting diode with a first set of electrical conductors associated with the endotracheal tube;
(d) further providing power to the camera with a second set of electrical conductors associated with the endotracheal tube;
(e) conveying a signal reflective of the image captured by the camera with the second set of electrical conductors;
(f) detachably attaching the terminal ends of the first and second sets of electrical conductors with a source of electrical power in a transmitter module;
(g) further conveying the signal to a transmitter in the transmitter module;
(h) transmitting the signal to a receiver; and
(i) displaying an image reflective of the signal on a video monitor.

13. The method as set forth in claim 12 wherein said steps of providing and further providing provide electrical power to the first and second set of conductors extending from within the side wall of the endotracheal tube.

14. The method as set forth in claim 13 wherein said step of conveying provides the signal to the second set of conductors extending from within the side wall of the endotracheal tube.

15. The method as set forth in claim 12 including the step of carrying out said step of detachably attaching at a location remote from the endotracheal tube.
